(19) United States
(12) Patent Application Publication
(10) Pub. No.: US 2011/0230684 A1
(43) Pub. Date: Sep. 22, 2011

(54) PROCESS AND APPARATUS FOR PRODUCING EHTYLENICALLY UNSATURATED HALOGENATED HYDROCARBONS

(76) Inventors: Michael Benje, Bad Soden (DE); Peter Kammerhofer, Burgkirchen (DE); Klaus Krejci, Burghausen (DE); Rainer Kampschulte, Burghausen (DE); Helmut Grumann, Perach (DE)

(21) Appl. No.: 12/998,174

(22) PCT Filed: Sep. 3, 2009
(86) PCT No.: PCT/EP2009/006383

§ 371 Date: Jun. 6, 2011

(30) Foreign Application Priority Data
Sep. 26, 2008 (DE) 10 2008 049 261.2

(51) Int. Cl. C07C 17/23; B01J 19/00; G05D 7/00; B01J 19/12

(52) U.S. Cl. 570/226; 422/198; 422/111; 422/108; 204/157.98; 204/157.61

(57) ABSTRACT

The invention relates to a process and an apparatus for product-conserving thermal dissociation of halogenated aliphatic hydrocarbons, preferably for thermal dissociation of 1,2-dichloroethane to vinyl chloride. This uses chemical dissociation promoters and/or physical measures which initiate the dissociation reaction. The initiation of the dissociation reaction, with the same conversion, lowers the temperature level in the reaction mixture and the temperature of the dissociation gas on exit from the dissociation furnace. The amount and the exit temperature of the flue gas from the radiation zone of the dissociation furnace likewise decrease at the same time. It order to be able to implement the product-conserving mode of operation in the radiation zone of the dissociation furnace and simultaneously to maintain the function of the convection zone, the heat input to the dissociation furnace is divided such that a portion of the heat introduced by underfiring is introduced by burners in the radiation zone, and the other portion of the heat supplied by underfiring is introduced by burners arranged at the exit of the flue gas from the radiation zone.

The partial decoupling of the heat input of the radiation zone and of the convection zone makes possible a particularly product-conserving mode of operation.

### CLAIM FOR PRIORITY

This substitute specification is submitted as a national phase entry of International Patent Application No. PCT/EP2009/006383 (International Publication No. WO 2010/034396), filed Sep. 3, 2009, entitled “Method and Device for Producing Ethylenically Unsaturated Halogenated Hydrocarbons” which claims priority to German Patent Application No. DE 10 2008 049 261.2, filed Sep. 26, 2008 and is entitled, “Verfahren Und Vorrichtung Zur Herstellund Von Ethylenisch Ungesättigten Halogenierten Kohlenwasserstoffen”. The priorities of International Patent Application No. PCT/EP2009/006383 and German Patent Application No. DE 10 2008 049 261.2 are hereby claimed and their disclosures incorporated herein by reference in their entireties.

The present invention relates to a particularly product-conserving process and an apparatus suitable therefor for preparing ethylenically unsaturated halogen compounds by thermal dissociation of halogenated aliphatic hydrocarbons, in particular the preparation of vinyl chloride by thermal dissociation of 1,2-dichloroethane.

The invention is described below by way of example for the production of vinyl chloride (hereinafter referred to as VCM) by thermal dissociation of 1,2-dichloroethane (hereinafter referred to as EDC), but can also be used for the preparation of other ethylenically unsaturated halogen compounds.

Terminology used herein is given its ordinary meaning unless otherwise stated herein.

### BRIEF DESCRIPTION OF THE DRAWINGS

The invention is described in detail below with reference to the drawings wherein like numbers designate similar parts and wherein:

FIG. 1 is a schematic of a reactor for producing ethylenically unsaturated halogenated hydrocarbons for halogenated aliphatic hydrocarbons as described herein.

FIG. 1A is a schematic of an older style of reactor for producing ethylenically unsaturated halogenated hydrocarbons for halogenated aliphatic hydrocarbons retrofitted to accept the invention as described herein.

FIG. 2 is a schematic of the integration of the reactor of FIG. 1 into a system for producing ethylenically unsaturated halogenated hydrocarbons for halogenated aliphatic hydrocarbons as described herein.

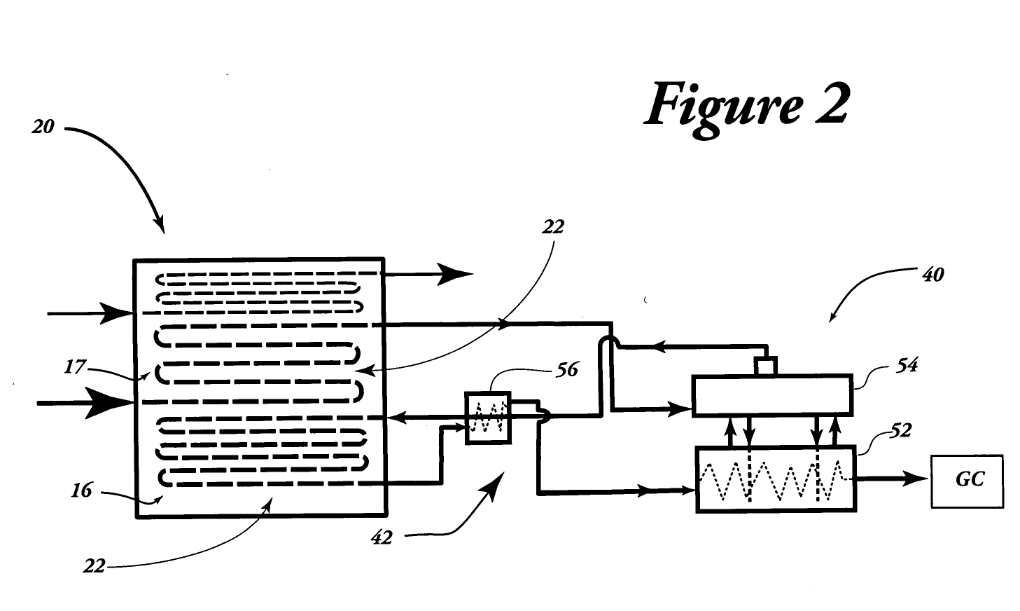

### DETAILED DESCRIPTION

VCM is nowadays prepared predominantly by thermal dissociation of EDC, with the reaction being carried out industrially according to the equation

C₂H₄Cl₂+71 kJ->C₂H₃Cl+HCl

in a reaction tube 22 which is in turn located in a gas- or oil-heated furnace 20.

The reaction is usually allowed to proceed to a conversion of 55-65%, based on the EDC used (hereinafter feed EDC). The temperature of the reaction mixture leaving the furnace (hereinafter furnace exit temperature) is about 480-520° C. The reaction is carried out under superatmospheric pressure. Typical pressures at the furnace inlet are about 13-30 bar abs. in present-day processes.

At higher conversions and, resulting therefrom, a higher partial pressure of VCM in the reaction mixture, VCM is increasingly converted under the reaction conditions into subsequent products such as acetylene and benzene which in turn are precursors of carbon deposits. The formation of carbon deposits makes shutdown and cleaning of the reactor at regular intervals necessary. In view of this, a conversion of 55%, based on the EDC used, has been found to be particularly advantageous in industrial practice.

The majority of processes employed at present operate using cuboidal furnaces 20 in which the reaction tube 22 is arranged centrally as a serpentine tube made up of horizontal tubes 22a, 22s, 22b arranged vertically above one another, with the serpentine tube 22 being able to have a single or double configuration. In the case of a single configuration, the tubes can either be aligned or offset. The furnaces 20 are heated by means of burners 26, 28 which are arranged in rows in the furnace walls 24. The transfer of heat to the reaction tubes 22b occurs predominantly by wall and gas radiation but also convectively via the flue gas 38 formed in heating by means of burners. The dissociation of EDC is sometimes also carried out in other types of furnace 20 having a different arrangement of the reaction tubes 22 and the burners 26.

The invention can in principle be applied to all types of furnace 20 and burner 26, 28 arrangements and also to other ways of heating the reaction.

A typical tube reactor used for the dissociation of EDC comprises a furnace 20 and a reaction tube 22. In general, such a furnace 20 fired by means of a primary energy carrier, e.g. oil or gas, is divided into a radiation zone 16 and a convection zone 17.

In the radiation zone 16, the heat required for the dissociation is transferred to the reaction tube 22b primarily by radiation from the burner-heated furnace walls 24 and the hot flue gas 38.

In the convection zone 17, the energy content of the hot flue gases 38 leaving the radiation zone 16 is utilized by convective heat transfer. In this way, the starting material for the dissociation reaction, e.g. EDC, can be preheated, vaporized or superheated. The generation of steam and/or the preheating of combustion air is likewise possible.

In a typical arrangement as described, for example, in EP 264,065 A1 (incorporated herein by reference in its entirety), liquid EDC is firstly preheated in the convection zone 17 of the dissociation furnace 20 and then vaporized in a specific vaporizer 40 outside the dissociation furnace 20. The gaseous EDC is then fed into the convection zone 17 again and superheated there, with the dissociation reaction being able to commence here. After superheating has occurred, the EDC enters the radiation zone 16 where the conversion into vinyl chloride and hydrogen chloride takes place.

The burners 26 are usually arranged in superposed rows on the longitudinal sides and end faces of the furnace 20, with efforts being made by means of the type and arrangement of the burners 26 to achieve very uniform distribution of inward radiation of heat along the circumference of the reaction tubes 22.

The part of the furnace 20 in which the burners 26 and the reaction tubes 22b are arranged and in which the predominant conversion of the dissociation reaction takes place is referred to as the radiation zone 16. Above the actual reaction tubes 22b and upstream of the radiation zone 16 as seen in the flow direction of the reaction mixture there are usually further rows of tubes 22s which are preferably composed of tubes 22s arranged horizontally next to one another. These rows of tubes 22s are typically unfinned and largely shield internals 22a located above them, e.g. finned heat exchange tubes 22a of the convection zone 17, against direct radiation from the firing space. In addition, these rows of tubes 22s increase the thermal efficiency of the reaction zone by means of structurally optimized convective heat transfer. In technical language usage, these tubes or rows of tubes 22s are usually referred to as “shock tubes” or “shock zone”.

For the purposes of the invention, the “reaction zone” is made up of the reaction tubes 22b which are located downstream of the shock zone in the flow direction of the reaction gas and are preferably vertically aligned or offset above one another. The major part of the EDC used is converted into VCM here.

The actual dissociation reaction takes place in the gaseous state. Before entering the reaction zone, the EDC is firstly preheated and then vaporized and possibly superheated. Finally, the gaseous EDC enters the reactor where it is usually heated further in the shock tubes 22s and finally enters the reaction zone where the thermal dissociation reaction commences at temperatures above about 400° C.

The heat of the hot flue gas 38 leaving the radiation zone 16 is utilized by convective heat transfer in the convection zone 17 which follows the radiation zone 16 and is physically located above the latter, with, for example, the following operations being able to be carried out:
preheating of liquid EDC
vaporization of preheated EDC
heating of heat transfer media
preheating of boiler feed water
generation of steam
preheating of combustion air
preheating of other media (including media extraneous to the process).

Vaporization of EDC in the tubes 22a located in the convection zone 17 is dispensed with in modern plants since in this mode of operation the vaporizer tubes 22a quickly become blocked by carbon deposits, which adversely affects the economics of the process as a result of shortened cleaning intervals.

The physical combination of radiation zone 16 and convection zone 17 with the associated flue gas chimney 36 is referred to as dissociation furnace 20 by those skilled in the art.

The utilization of the heat content of the flue gas 38, for example for preheating the EDC, is of central importance for the economics of the process since very complete exploitation of the heat of combustion of the fuel has to be sought.

The reaction mixture leaving the dissociation furnace 20 contains not only the desired product VCM but also HCl (hydrogen chloride) and unreacted EDC. These are separated off in subsequent process steps and recirculated to the process or utilized further. Furthermore, the reaction mixture contains by-products which are likewise separated off, worked up and utilized further or recirculated to the process. These relationships are known to those skilled in the art.

The by-products carbon and tar-like substances which are formed over a plurality of reaction steps from low molecular weight by-products such as acetylene and benzene and deposit in the serpentine tubes 22a of the dissociation furnace 20 (and also in downstream apparatuses such as the EDC vaporizer) where they lead to a deterioration in heat transfer and, by constricting the free cross section, to an increase in the pressure drop are of particular importance for the process.

These by-products lead to the plants having to be shutdown and cleaned at regular intervals. Owing to the high costs for the cleaning itself and also the associated loss of production, very long time intervals between cleaning operations are desired.

After exit from the dissociation furnace 20, the sensible heat of the dissociation gas can be utilized for vaporizing the feed EDC.

Apparatuses for this purpose are described, for example, in EP 264,065 A1 or DE 36 30 162 A1. An apparatus corresponding to EP 264,065 A1, in which the feed EDC is vaporized outside the furnace 20 by means of the sensible heat content of the dissociation gas, has been found to be particularly advantageous.

Directly after the utilization of heat by vaporization of feed EDC and cooling of the dissociation gas (in the case of processes in which the heat of the dissociation gas is not recovered, also directly after exit from the dissociation furnace 20), the dissociation gas is scrubbed and cooled further in a quenching column by direct contact with a cool, liquid runback stream or circulated stream. This has the primary purpose of scrubbing out carbon particles present in the dissociation gas or condensing and likewise scrubbing out tar-like substances which are still gaseous since both components would interfere in the subsequent work-up steps.

Finally, the dissociation gas is passed to a work-up by distillation, in which the components hydrogen chloride (HCl), VCM and EDC are separated from one another.

This work-up stage generally comprises at least one column which is operated under superatmospheric pressure and in which pure HCl is obtained as overhead product (hereinafter HCl column).

Attempts have for a long time been made to increase the space-time yield of the EDC dissociation by means of various measures. These measures have the aim of increasing the amount of product which can be obtained from a given reactor volume and can be divided into:
use of heterogeneous catalysts
use of chemical promoters
other measures (e.g. injection of electromagnetic radiation).

It is generally assumed that the measures proposed hitherto contribute to physical or chemical initiation to provide free chlorine radicals in the reaction space. The thermal dissociation of EDC is a free-radical chain reaction in which the first step is elimination of a free chlorine radical from an EDC molecule:

C₂H₄Cl₂->C₂H₄Cl+Cl

The high activation energy of this first step compared to the subsequent chain propagation steps is the reason why the dissociation reaction proceeds appreciably only above a temperature of about 420° C.

The use of a heterogeneous catalyst makes elimination of a free chlorine radical from the EDC molecule possible, e.g. by dissociative adsorption of the EDC molecule on the catalyst surface. Very high EDC conversions can be achieved using heterogeneous catalysts. However, decomposition of the VCM and thus carbon formation on the catalyst surface occur on and in the vicinity of the catalyst surface as a result of high local partial pressures of VCM, leading to rapid deactivation of the catalyst. Owing to the frequent regenerations made necessary thereby, heterogeneous catalysts have hitherto not been employed in the large-scale production of VCM.

In the case of physical measures, e.g. irradiation with short-wavelength light, the energy for elimination of the free chlorine radical is provided from an external source.

Thus, adsorption of a quantum of short-wavelength light by the EDC molecule provides the energy for elimination of the free chlorine radical:

C₂H₄Cl₂+hv->C₂H₄Cl+Cl

where “v” indicates the frequency of a photon. When chemical initiators are used, a chlorine atom is eliminated from the EDC molecule by reaction of the EDC with the initiator or the free chlorine radicals are provided by decomposition of the initiator. Chemical initiators are, for example, elemental chlorine, bromine, iodine, elemental oxygen, chlorine compounds such as carbon tetrachloride (CCl₄) or chlorine-oxygen compounds such as hexachloroacetone.

All the measures for initiating the reaction bring about a significant reduction in the temperature level in the reactor at a given conversion or a large increase in the conversion at a given temperature level.

Comprehensive literature is available on the use of catalysts for thermal dissociation of EDC. An example which may be mentioned is EP 002,021 A1.

The high tendency of catalysts to become carbonized and the need for frequent regeneration stand in the way of the use of these in industrial practice.

Physical measures such as injection of electromagnetic radiation into the reaction tube (described, for example, in DE 30 08 848 A1 or DE 29 38 353 A1) have also not found their way into industrial practice despite their suitability in principle. The reasons for this may well be related to safety (since, for example, a pressure-resistant optical window is necessary for input of light). Further physical measures which have been described, for instance injection of a heated gas into the reaction mixture (WO 02/094,743 A2) have also not been used hitherto on an industrial scale.

DE 103 19 811 A1 describes the electromagnetic and photolytic induction of free-radical reactions. In addition, this document describes an apparatus for introducing this energy into a reactor. Although this document mentions the use of dissociation promoters in general terms, no information can be found there about the design and the operation of the reactor used.

The use of chemical promoters is in principle the least technically complicated because it is neither necessary to fill the reactor with catalyst (facilities for filling/emptying and regeneration are required) nor are additional facilities for injection of electromagnetic radiation required. The promoter can be introduced into the feed EDC stream in a simple manner.

Increasing the conversion of the EDC dissociation by addition of halogens or halogen-releasing compounds has been described by Barton et al. (U.S. Pat. No. 2,378,859 A), where the experiments of fundamental importance were carried out at atmospheric pressure in a glass apparatus. Krekeler (DE patent No. 857,957) has described a process for the thermal dissociation of EDC under superatmospheric pressure. Carrying out the reaction at superatmospheric pressure is of fundamental importance for large-scale industrial use since only then is economical fractionation of the reaction mixture possible. This relationship is known to those skilled in the art. Krekeler also recognized the problem of accelerated formation of carbon deposits at high conversions and nominated 66% as a practical upper limit to the conversion. In DE-B-1,210,800, Schmidt et al. describe a process in which operation at superatmospheric pressure is combined with the addition of a halogen. Here, conversions of about 90% are achieved at working temperatures of 500-620° C. Schmidt et al. also stated that the conversion reaches saturation as a function of the amount of halogen added, i.e. that a significant increase in conversion is no longer achieved above a particular amount of halogen added relative to the feed EDC stream.

The simultaneous addition of halogen or other chemical promoters at least two points on the reactor tube has been described by Sonin et al. in DE 1 953 240 A. Here, conversions in the range from 65 to 80% were achieved at reaction temperatures of 250-450° C.

In DE 2 130 297 A, Scharein et al. describe a process for the thermal dissociation of EDC under superatmospheric pressure, in which chlorine is introduced at a plurality of points on the reactor tube 22. Here, conversion of 75.6% (example 1) or 70.5% (example 2) are achieved at a reaction temperature of 350-425° C. This publication also refers to the importance of the ratio of surface area/volume of the reactor and to the importance of the loading of the heating areas (heat flux).

The problem of rapid carbonization of the reactor at high conversions of the dissociation reaction is avoided in a process disclosed by Demaiziere et al. in U.S. Pat. No. 5,705,720 A by diluting the gaseous EDC entering the reactor with hydrogen chloride. Here, hydrogen chloride is added to the EDC in a molar ratio of from 0.1 to 1.8. At the same time, dissociation promoters can also be added to the mixture of EDC and HCl according to this process. Since the VCM partial pressure is kept low by the dilution with large amounts of HCl, high conversions can be achieved without carbonization of the reactor. However, disadvantages here are the energy input for heating and the subsequent removal of the HCl added for dilution.

In U.S. Pat. No. 4,590,318 A, Longhini discloses a process in which a promoter is introduced into the dissociation gas after exit from the dissociation furnace, i.e. into the after-reaction zone 42. Here, the heat content of the dissociation gas is exploited in order to increase the total conversion of the EDC dissociation. However, this method is inferior to measures for increasing the space-time yield in the dissociation furnace 20 itself since only the heat still present in the dissociation gas stream after exit from the dissociation furnace 20 can be exploited and the usable quantity of heat is limited when the heat of the dissociation gas stream is to be utilized for vaporization of the feed EDC.

Felix et al. (EP 0 133 699 A1), Wiedrich et al. (U.S. Pat. No. 4,584,420 A) and Mielke (DE 42 28 593 A1) teach the use of chlorinated organic compounds instead of chlorine as dissociation promoters. This makes it possible in principle to achieve the same effects on the EDC dissociation reaction as when using elemental halogens such as chlorine or bromine. However, since these are materials which are not frequently available like chlorine in the integrated facility for VCM production, they have to be introduced separately into the process, which in turn is associated with increased costs for procurement and disposal of the residues.

DE 102 19 723 A1 relates to a process for metered addition of dissociation promoters in the course of preparation of unsaturated halogenated hydrocarbons. This document does not disclose any further details regarding the thermal design of the reactor.

Although the effects of dissociation promoters on the reaction of thermal dissociation of EDC and their main advantages have been known for a relatively long time, the use of dissociation promoters has hitherto not found its way into the commercial production of VCM by thermal dissociation.

This is because all previously disclosed processes aim at increased conversions of the dissociation reaction (at least 65%), although it was recognized early on (DE patent 857 957) that a significantly increased tendency for carbon deposits to be formed in the reactor tubes 22 and the after-reaction zone 42 has to be expected above this limit. The increased tendency for formation of carbon deposits, which has hitherto prevented the use of dissociation promoters in industrial practice is due not to the promoters themselves but to a combination of higher VCM partial pressures in the reaction mixture (as occur at conversions above 65%) with high temperatures of the dissociation gas and the interior wall of the reactor tube 22. This hypothesis is also supported, in particular, by the results disclosed in U.S. Pat. No. 5,705,720 A where high conversions can be achieved with and without dissociation promoter by dilution of the reaction mixture with relatively large amounts of HCl, without an increased tendency for carbon deposits to be formed occurring.

DE 103 26 248 A1 describes energy optimization in the preparation of vinyl chloride by dissociation of DCE, and exploitation of the energy contents in the offgas stream. This document describes neither the use of dissociation promoters nor of electromagnetic radiation. Nor does this document contain any indication to the combination of measures a) to d) described below.

DE 19 08 624 A discloses a tube oven for thermal dissociation of hydrocarbons. Use of dissociation promoters or of locally limited energy supply is not described.

It has now been found that, using chemical dissociation promoters or physical methods to initiate the dissociation reaction makes it possible to achieve a particularly product-conserving process for the thermal dissociation of EDC.

It is an object of the present invention to provide a reactor in which the thermal dissociation of halogenated aliphatic hydrocarbons can be achieved at substantially lower temperatures but with comparable efficiency compared to conventional plants.

A further object of the present invention is to provide a process for the thermal dissociation of halogenated aliphatic hydrocarbons, in which significantly lower temperatures can be employed compared to conventional processes, without any adverse effect on the efficiency of the process.

The invention provides a process for the thermal dissociation of halogenated aliphatic hydrocarbons to form ethylenically unsaturated halogenated hydrocarbons in a reactor which comprises reaction tubes 22 running through a convection zone 17 and through a radiation zone 16 located downstream in the flow direction of the reaction gas, wherein
a) a chemical promoter for the thermal dissociation is introduced into the reaction tubes 22 and/or localized energy input to form free radicals into the reaction tubes 22 is effected at one or more points within the reactor 20,
b) a portion of the total amount of heat energy introduced by combustion is introduced by burners 26 in the radiation zone 16 of the dissociation furnace 20,
c) the remaining portion of the total amount of heat energy introduced by combustion is introduced by burners 28 which heat the space upstream of the radiation zone 16 viewed in flow direction of the reaction gas, and
d) the conversion of the dissociation reaction, based on the halogenated aliphatic hydrocarbon used, is in the range from 50 to 65%, preferably from 52 to 57%.

The invention further provides an apparatus for the thermal dissociation of halogenated aliphatic hydrocarbons to form ethylenically unsaturated halogenated hydrocarbons, which comprises a reactor 20 which comprises reaction tubes 22 running through a convection zone 17 and through a radiation zone 16 located downstream in the flow direction of the reaction gas, comprising the elements:
A) means 44 of introducing chemical promoters for the thermal dissociation into the reaction tubes 22 and/or means 46 of introducing localized energy to form free radicals in the reaction tubes 22,
B) one or more burners 26 which fire the reaction tubes 22 in the radiation zone 16, and
C) one or more burners 28 which fire the reaction tubes 22 in the convection zone 17.

Under these conditions, even in the case of use of a chemical promoter and/or of a physical measure for free radical formation, the by-product formation caused by these measures themselves is more than compensated by the lowering of the general formation rate of by-products owing to the lowering of the temperature in the reaction mixture (dissociation gas). As a consequence, this gives rise to significantly lower coke formation rates and hence longer cleaning intervals than in the case of conventional processes. This effect is particularly marked in the case of use of elemental chlorine as a promoter.

Since, in the process of the invention, the radiation zone 16 of the dissociation furnace 20 is heated less strongly than in the case of noninventive processes, both the amount and temperature of the flue gas 38 which emerges from the radiation zone 16 are lower than in previously known processes. Due to the specified reduction in the energy introduced by reduced combustion in the radiation zone 16, sufficient heat is therefore no longer available in order to fulfill the tasks of the convection zone 17 in terms of process technology, principally the preheating of EDC.

This problem is solved in accordance with the invention by generating a portion of the total heat energy introduced by combustion by means of burners 26 in the radiation zone 16 of the dissociation furnace 20, and generating the remaining portion by means of burners 28 arranged in the convection zone 17, preferably at the flue-gas-side entry into the convection zone 17. These burners 28 are particularly preferably arranged above the shock tubes 22s.

The gas supply to the burners 26 of the radiation zone 16 and the burners 28 at the flue-gas-side entry into the convection zone 17 is preferably regulable separately.

In a preferred embodiment of the process according to the invention, as additional measure (=measure e), the dew point of the flue gas 38 is determined at the exit 36 from the convection zone 17 or in the flue gas chimney 37, and this serves as command variable for the regulation of the amount of fuel and/or for the regulation of the amount of the chemical promoter added and/or for the regulation of the intensity of the localized energy input.

DE 22 35 212 A describes an improved measuring instrument for the monitoring of the dew point of flue gases. This does not incite the person skilled in the art to use this instrument in processes for thermal dissociation of saturated halogenated hydrocarbons and especially not in conjunction with the use of dissociation promoters and/or locally limited energy inputs.

In a further preferred embodiment of the process of the invention, the flue gas 38 is condensed in a heat exchanger 50 and the waste heat from the flue gas 38 is utilized for preheating the burner air, as additional measure (=measure f).

In the process variant comprising measure f), the heat from the cooling of the flue gas 38 to below its dew point and the heat of condensation of the flue gas 38 are utilized.

In the case of measure f), heat exchange preferably occurs at the point at which the flue gas 38 leaves the convection zone 17.

The process of the invention may comprise measures e) or f) or a combination of measures e) and f).

The process of the invention preferably comprises measure e).

Measure e) is employed especially in the case of fuels with a moderate or high proportion of acid-forming components. However, this measure can also be used in the case of fuels with a low proportion of acid-forming components.

Measure f) is employed especially in the case of fuels having a low proportion of acid-forming components. However, this measure can also be used in the case of fuels having a moderate or high proportion of acid-forming components.

Apparatuses in which the process of the invention comprising measure e) is carried out comprise as additional elements:
D) means DP of determining the dew point of the flue gas 38 at the exit 36 from the convection zone 17 or in the flue gas chimney 37, and
E) means (V, 44, 46 respectively) of regulating the amount of fuel and/or of regulating the amount of the chemical promoter added and/or of regulating the intensity of the localized energy input, the dew point of the flue gas 38 at the exit 36 from the convection zone 17 or in the flue gas chimney 37 serving as a command variable for the regulation.

Apparatuses in which the process of the invention comprising measure f) is carried out comprise as additional element:
F) at least one heat exchanger 50 for recovering waste heat from the condensation of the flue gas for preheating the combustion air or other media, e.g. EDC.

The process of the invention is described by way of example for the EDC/VC system. It is also suitable for preparing other halogen-containing unsaturated hydrocarbons from halogen-containing saturated hydrocarbons. In all these reactions, the dissociation is a free-radical chain reaction in which not only the desired product but also undesirable by-products which on long-term operation lead to carbon deposits in the plants are formed. Preference is given to the preparation of vinyl chloride from 1,2-dichloroethane.

For the purposes of the present description, “localized energy input to form free radicals in the reaction tubes” refers to physical measures which are able to initiate the dissociation reaction. Such measures can be, for example, injection of high-energy electromagnetic radiation or local introduction of thermal or nonthermal plasmas, e.g. hot inert gases.

Means 44 of introducing chemical promoters for the thermal dissociation together with the halogenated aliphatic hydrocarbon into the reaction tubes 22 are known to those skilled in the art. These can be feed lines 45 which allow introduction of predetermined amounts of chemical promoters into the feed gas stream, which is then fed to the reactor 20. However, they can also be feed lines 44 which allow the introduction of predetermined amounts of chemical promoters into the reaction tubes 22, for example at the level of the convection zone 17 and/or at the level of the radiation zone 16. These feed lines can have nozzles at the reactor end. Preference is given to one or more of these feed lines opening into the tubes 22 in the radiation zone 16, very particularly preferably in the first third, viewed in the flow direction of the reaction gas, of the radiation zone 16.

Means 46 of introducing localized energy into the reaction tubes 22 to form free radicals are likewise known to those skilled in the art. These can likewise be feed lines 47 which may have nozzles at the reactor end and via which thermal or nonthermal plasma is introduced into the reaction tubes 22 at the level of the convection zone 17 and/or at the level of the radiation zone 16; or they can be windows via which electromagnetic radiation or particle beams are injected into the reaction tubes 22. The feed lines 47 or windows can open into the reaction tubes 22 at the level of the convection zone 17 and/or at the level of the radiation zone 16.

Preference is given to one or more of these feed lines 45, 47 opening into the tubes 22 in the first third, viewed in the flow direction of the reaction gas, of the radiation zone 16; or the windows for injection of the radiation being installed in the first third of the radiation zone 16.

The amount of the chemical promoter and/or the intensity of the localized energy input should be selected in the individual case such that the desired molar conversion of the dissociation reaction is also achieved at the given internal reactor temperature.

Ways of selecting the amount of the chemical promoter and/or the intensity of the localized energy input into the reaction tubes to form free radicals are likewise known to those skilled in the art. These are generally regulating circuits in which a command variable is used to regulate the amount or intensity. As command variables, it is possible to use all process parameters by means of which it is possible to draw conclusions as to the molar conversion of the dissociation process. Examples are the temperature of the exiting reaction gases, the content of dissociation products in the reaction gases or the wall temperature of the reaction tubes 22 at selected places.

The above-described combination of measures or features make greatly reduced internal reactor temperatures, compared to conventional processes or apparatuses, possible without any adverse effect on the conversion of the dissociation reaction. The disadvantages known from the literature, e.g. increased formation of by-products and a strong tendency for carbon deposits to be formed, can thereby be avoided.

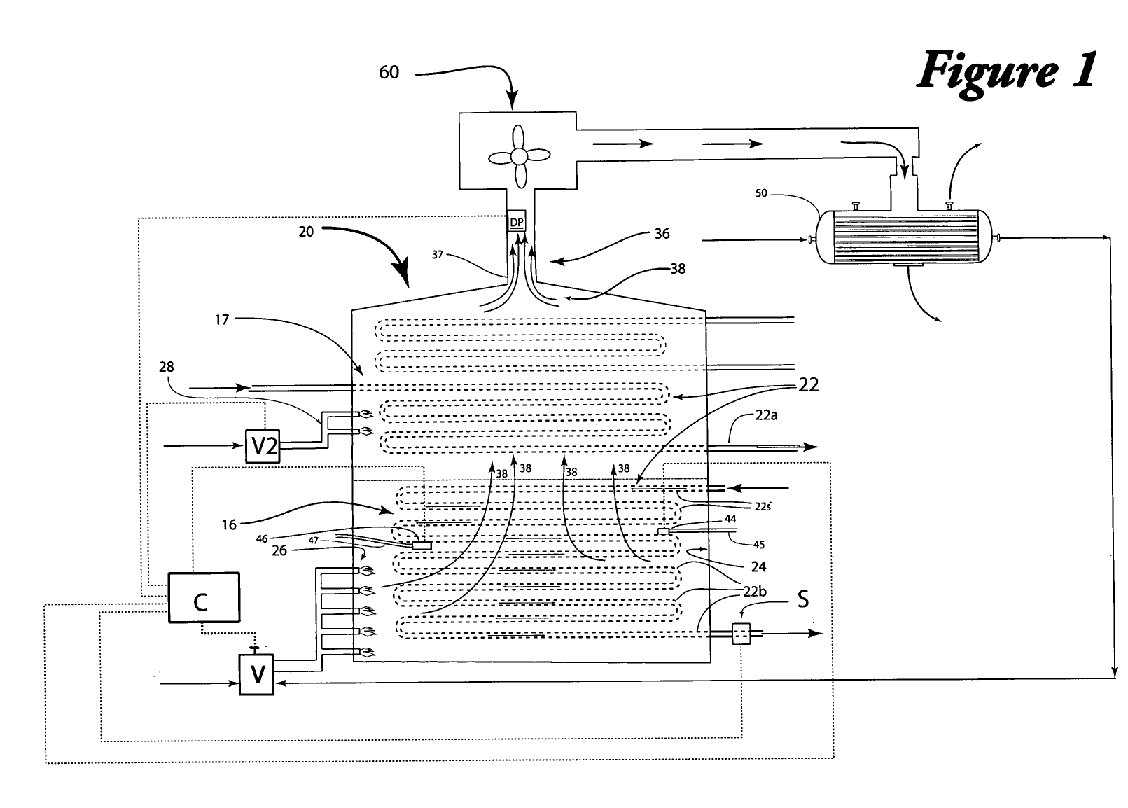

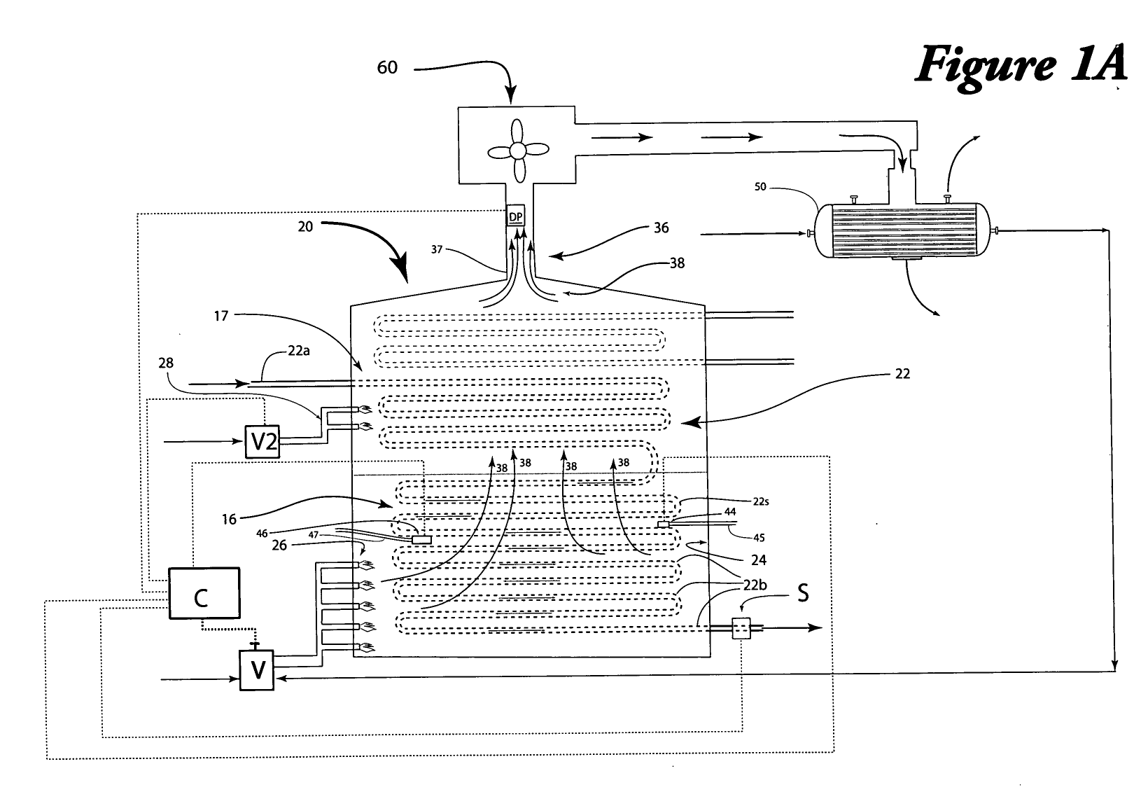

In a preferred variant of the process of the invention as illustrated in FIG. 1A, at one or more points on the shock tubes 22s or the tubes 22b in the reaction zone, electromagnetic radiation of a suitable wavelength or a particle beam is radiated in or a chemical promoter is added or a combination of these measures is undertaken. In the case of the addition of a chemical promoter, the addition can preferably also be into the feed line 22s for the gaseous feed, for example into the EDC from the EDC vaporizer, before entry into the dissociation furnace 20.

The localized energy input to form free radicals is preferably effected by electromagnetic radiation or particle beams; particular preference is given here to ultraviolet laser light.

In the case of addition of a chemical promoter, the use of elemental halogen, in particular elemental chlorine, is preferred.

The chemical promoter can be diluted with a gas which is inert toward the dissociation reaction, with the use of hydrogen chloride being preferred. The amount of inert gas used as diluent should not exceed 5 mol % of the feed stream.

The intensity of the electromagnetic radiation or the particle beam or the amount of the chemical promoter is set so that, at the intended internal reactor temperature, the molar conversion, based on the feed, at the dissociation gas-end outlet of the feed vaporizer is in the range from 50 to 65%, preferably from 52 to 57%.

Particular preference is given to a molar conversion, based on the EDC used, at the dissociation gas-end outlet of the feed vaporizer of 55%.

The temperature of the reaction mixture leaving the reactor 20 is preferably in the range from 400° C. to 470° C.

The process of the invention is particularly preferably used for the thermal dissociation of 1,2-dichloroethane to form vinyl chloride.

In a preferred variant of the invention, not only the thermal dissociation of halogenated, aliphatic hydrocarbons in the actual dissociation furnace but also, as further process step, the vaporization of the liquid feed, for example the liquid EDC, before entry into the radiation zone of the dissociation furnace, is undertaken. These measures have a positive influence on the economics of the dissociation process and the operation of the dissociation furnace.

A preferred embodiment of the invention is directed to a process in which the sensible heat of the dissociation gas is exploited in order to vaporize liquid, preheated feed, e.g. EDC, before entry into the radiation zone, preferably using a heat exchanger as has already been described in EP 276,775 A2, incorporated herein by reference in its entirety. Particular attention should here be given to ensuring that firstly the dissociation gas is still hot enough on leaving the dissociation furnace 20 to vaporize the total amount of the feed by means of its sensible heat content and secondly the temperature of the dissociation gas on entering this heat exchanger does not go below a minimum value in order to prevent condensation of tar-like substances in the heat exchanger tubes 22.

In a further preferred embodiment of the vaporization of the feed, which has likewise been described in EP 276,775 A2, the temperature of the dissociation gas at the exit from the dissociation furnace is so low that the heat content of the dissociation gas is not sufficient to vaporize the feed completely. In this embodiment of the invention, the missing proportion of gaseous feed is produced by flash evaporation of liquid feed in a vessel, preferably in the steaming-out vessel of a heat exchanger, as has been described in EP 276,775 A2. In this case, preheating of the liquid feed advantageously occurs in the convection zone 17 of the dissociation furnace 20. In this embodiment of the invention, too, it has to be ensured that the temperature of the dissociation gas at the inlet into this heat exchanger does not go below a minimum value in order to prevent condensation of tar-like substances in the heat exchanger tubes 22.

The heat content of the dissociation gas is used in this preferred process variant to vaporize at least 80% of the feed by means of indirect heat exchange without the dissociation gas condensing either partly or completely.

As heat exchanger, preference is given to using an apparatus as is described, for example, in EP 264,065 A1 incorporated herein in its entirety. Here, liquid halogenated aliphatic hydrocarbon is heated indirectly by the hot product gas comprising the ethylenically unsaturated halogenated hydrocarbon which leaves the reactor 20, vaporized and the resulting gaseous feed gas is introduced into the reactor 20, with the liquid halogenated aliphatic hydrocarbon being heated to boiling by the product gas in a first vessel 52 and from there being transferred to a second vessel 54 in which it is partly vaporized without further heating under a pressure which is lower than in the first vessel 52 and the vaporized feed gas being fed into the reactor 20 and the unvaporized halogenated aliphatic hydrocarbon being recirculated to the first vessel 52.

In a particularly preferred variant of this process, the halogenated aliphatic hydrocarbon is heated in the convection zone 17 of the reactor 20 by means of the flue gas 38 produced by the burners 26, 28 which heat the reactor 20 before being fed into the second vessel 54.

Particular preference is given to a mode of operation in which the entire feed is vaporized by indirect heat exchange with the dissociation gas without the dissociation gas being either partly or completely condensed.

If the feed is not vaporized completely by means of the heat content of the dissociation gas, the residual amount of feed is preferably vaporized by flush evaporation into a vessel, with the feed being preheated beforehand in the liquid state in the convection zone 17 of the dissociation furnace 20. As vessel for the flash evaporation, preference is given to using the flash evaporator of a heat exchanger, as has been described, for example, in EP 264,065 A1.

In a further preferred variant of the process of the invention, the temperature of the reaction gas entering the heating apparatus 40 located outside the reactor is measured and serves as command variable for regulation of the amount of chemical promoter added and/or the intensity of the localized energy input. Of course, other measured parameters can also be employed as command variable, for example the content of products of the dissociation reaction.

In a further preferred process variant, the molar conversion of the dissociation reaction is determined downstream of the point at which the dissociation gas leaves the EDC vaporizer 40 or at the top of the quenching column, for example by means of an on-line analytical apparatus, preferably by means of an on-line gas chromatograph GC.

In a further preferred variant of the process of the invention, the flue gas 38 is extracted by means of a flue gas blower 60 after leaving the convection zone 17 and is passed through one or more heat exchangers where it is condensed. The waste heat is utilized for heating the burner air. The condensate formed is, if appropriate, worked up and discharged from the process. The remaining gaseous constituents of the flue gas are, if appropriate, purified and released into the atmosphere.

Particular preference is given to a process in which the flue gas to be cooled to below the dew point is introduced in a downward direction from above into the heat exchanger provided for this purpose, after cooling leaves the heat exchanger in the upward direction and the condensate formed can freely runoff downward from the heat exchanger and is thus completely separated off from the flue gas stream.

The amount of fuel can be divided in either unequal parts or preferably equal parts over the burner rows in the furnace.

The economics of the process are also influenced by the sum of the pressure drops over the dissociation furnace 20 (comprising convection zone 17 and radiation zone 16), the heat exchanger 50 for vaporization of the feed and also any quenching system (“quenching column”) present. This should be as low as possible since when the dissociation products are separated off by distillation, they have to be condensed at the top of a column using a refrigeration machine for cooling the condenser. The greater the sum of the pressure drops over the total system for “thermal dissociation”, the lower the pressure of the top of the column and the dissociation product separated off, for example HCl, has to be condensed at a correspondingly lower temperature. This leads to an increased specific energy consumption by the refrigeration machine, which in turn has an adverse effect on the economics of the total process.

1-28. (canceled)

29. An improved process for the thermal dissociation of halogenated aliphatic hydrocarbons to form ethylenically unsaturated halogenated hydrocarbons in a heated reactor which comprises a reaction flow tube through which halogenated aliphatic hydrocarbon is passed, said reaction flow tube having a plurality of tube sections defined therein, said reactor further comprising a convection zone and a radiation zone located downstream of the convection zone when viewed in the flow direction of the reaction flow tube running through said convection zone and through said radiation zone, wherein the improvement comprises:
free radicals being formed in the reaction flow tube at one or more points within the reactor by:
introducing a chemical promoter for the thermal dissociation of halogenated aliphatic hydrocarbon into the reaction flow tube;
providing a localized energy input into the reaction tubes; or
both,
a portion of the total amount of heat energy required for thermal dissociation of said aliphatic halogenated hydrocarbon being introduced by burners in the radiation zone of the dissociation furnace,
another portion of the total amount of heat energy being introduced by burners which heat the space upstream of the radiation zone viewed in flow direction of the reaction gas in said reaction flow tube, and
the conversion of the dissociation reaction, based on the halogenated aliphatic hydrocarbon used is in the range from 50 to 65%;
and the temperature of the reaction mixture leaving the reactor is in the range from 400° C. to 470° C.

30. The process as claimed in claim 29, wherein the halogenated aliphatic hydrocarbon is 1,2-dichloroethane and the ethylenically unsaturated halogenated hydrocarbon is vinyl chloride.

31. The process as claimed in claims 30, wherein the energy content of the reaction gases leaving the radiation zone is used to heat the 1,2-dichloroethane in a heat exchange apparatus external to the reactor.

32. The process as claimed in claim 29, wherein the temperature of the reaction gas entering the heating apparatus located external the reactor is measured and serves as command variable for regulating the amount of the chemical promoter added and/or for the intensity of the localized energy input.

33. The process as claimed in claim 31, wherein: liquid 1,2-dichloroethane is indirectly heated and vaporized by the hot product gas comprising vinyl chloride which leaves the reactor, the resulting gaseous feed gas is introduced into the reactor, with the liquid 1,2-dichloroethane being heated to boiling by the product gas in a first vessel and from there being transferred to a second vessel in which it is partly vaporized without further heating under a pressure which is lower than in the first vessel and the vaporized 1,2-dichloroethane is fed into the reactor and the unvaporized 1,2-dichloroethane is recirculated to the first vessel.

34. The process as claimed in claim 33, wherein the 1,2-dichloroethane is heated by the flue gas produced by the burners which heat the reactor in the convection zone of the reactor before being fed into the second vessel.

35. The process as claimed in claim 29, wherein the 1,2-dichloroethane is heated by the flue gas produced by the burners which heat the reactor in the convection zone of the reactor before being fed into the second vessel.

36. The process as claimed in claim 30, wherein the molar conversion based on 1,2-dichloroethane is in the range from 52% to 57%.

37. The process as claimed in claim 30, wherein the localized energy input to form free radicals is effected by means of electromagnetic radiation or by means of a particle beam.

38. The process as claimed in claim 30, wherein free radicals are formed in the reaction flow tube by providing electromagnetic radiation in the form of ultraviolet laser light.

39. The process as claimed in claim 30, wherein free radicals are formed in the reaction flow tube by providing elemental chlorine is as a chemical promoter.

40. The process as claimed in claim 39, wherein the elemental chlorine is diluted with hydrogen chloride, the amount of the hydrogen chloride used for dilution being not more than 5 mol % of the halogenated aliphatic hydrocarbon stream used.

41. The process as claimed in claim 30, wherein the temperature of the reaction gas entering the heating apparatus located outside the reactor is measured and serves as command variable for regulating the amount of the chemical promoter added and/or for the intensity of the localized energy input.

42. The process as claimed in claim 29, wherein the localized energy input to form free radicals is effected by means of electromagnetic radiation or by means of a particle beam.

43. The process as claimed in claim 29, wherein free radicals are formed in the reaction flow tube by providing electromagnetic radiation in the form of ultraviolet laser light.

44. The process as claimed in claim 29, wherein free radicals are formed in the reaction flow tube by providing elemental chlorine is as a chemical promoter.

45. The process as claimed in claim 44, wherein the elemental chlorine is diluted with hydrogen chloride, the amount of the hydrogen chloride used for dilution being not more than 5 mol % of the halogenated aliphatic hydrocarbon stream used.

46. The process as claimed in claim 29, wherein the halogenated aliphatic hydrocarbon is heated in a heating apparatus arranged outside the reactor, using the energy content of the reaction gases leaving the radiation zone.

47. The process as claimed in claim 46, wherein the heating apparatus arranged outside the reactor comprises two vessels and wherein liquid halogenated aliphatic hydrocarbon is indirectly heated and vaporized by the hot product gas comprising the ethylenically unsaturated halogenated hydrocarbon which leaves the reactor, and the resulting gaseous feed gas is introduced into the reactor, with the liquid halogenated aliphatic hydrocarbon being heated to boiling by the product gas in the first vessel and from there transferred to the second vessel in which it is partly vaporized without further heating under a pressure which is lower than in the first vessel and the vaporized feed gas is fed into the reactor and the unvaporized halogenated aliphatic hydrocarbon is recirculated to the first vessel.

48. The process as claimed in claim 47, wherein the halogenated aliphatic hydrocarbon is heated by the flue gas produced by the burners which heat the reactor in the convection zone of the reactor before being fed into the second vessel.

49. The process as claimed in claim 29, wherein:
the halogenated aliphatic hydrocarbon is 1,2-dichloroethane and the ethylenically unsaturated halogenated hydrocarbon is vinyl chloride, and the temperature of the reaction mixture leaving the reactor is in the range from 400° C. to 470° C.;
the energy content of the reaction gases leaving the radiation zone is used to heat the 1,2-dichloroethane in an apparatus external to the reactor, said apparatus comprising two vessels wherein: liquid 1,2-dichloroethane is indirectly heated in the first vessel by the hot product gas comprising vinyl chloride which leaves the reactor, the resulting gaseous feed gas being introduced into the reactor, with the liquid 1,2-dichloroethane being heated to boiling by the product gas in the first vessel and from there being transferred to the second vessel in which it is partly vaporized without further heating under a pressure which is lower than in the first vessel and the vaporized 1,2-dichloroethane is fed into the reactor and the unvaporized 1,2-dichloroethane is recirculated to the first vessel; and
the molar conversion based on 1,2-dichloroethane is in the range from 52% to 57%.

50. The process as claimed in claim 49, wherein free radicals are formed in the reaction flow tube by providing electromagnetic radiation in the form of ultraviolet laser light.

51. The process as claimed in claim 49, wherein free radicals are formed in the reaction flow tube by providing elemental chlorine is as a chemical promoter.

52. The process as claimed in claim 49, wherein the elemental chlorine is diluted with hydrogen chloride, the amount of the hydrogen chloride used for dilution being not more than 5 mol % of the halogenated aliphatic hydrocarbon stream used.

53. The process as claimed in claim 49 wherein the localized energy input to form free radicals is effected by means of electromagnetic radiation or by means of a particle beam.

54. The process as claimed in claim 49, wherein free radicals are formed in the reaction flow tube by providing electromagnetic radiation in the form of ultraviolet laser light.

55. The process as claimed in claim 49, wherein the temperature of the reaction gas entering the heating apparatus located outside the reactor is measured and serves as command variable for regulating the amount of the chemical promoter added and/or for the intensity of the localized energy input.

56. The process as claimed in claim 49, wherein free radicals are formed in the reaction flow tube by providing elemental chlorine is as a chemical promoter.

57. The process as claimed in claim 56, wherein the elemental chlorine is diluted with hydrogen chloride, the amount of the hydrogen chloride used for dilution being not more than 5 mol % of the halogenated aliphatic hydrocarbon stream used.

58. The process as claimed in claim 49, further comprising the step of determining the dew point of the flue gas at the exit from the convection zone or in the flue gas chimney, and controlling the reaction by progress by: regulating the amount of fuel; regulating of the amount of the chemical promoter added; regulating the intensity of the localized energy input use based upon the measured dewpoint.

59. The process as claimed in claim 49, wherein the molar conversion of the dissociation reaction is determined downstream of the heating apparatus for the halogenated aliphatic hydrocarbon or at the top of the quenching column by means of an on-line gas chromatograph.

60. The process as claimed in claim 29, further comprising the step of determining the dew point of the flue gas at the exit from the convection zone or in the flue gas chimney, and controlling the reaction by progress by: regulating the amount of fuel; regulating of the amount of the chemical promoter added; regulating the intensity of the localized energy input use based upon the measured dewpoint.

61. The process as claimed in claim 29, wherein the halogenated aliphatic hydrocarbon is heated by the flue gas produced by the burners which heat the reactor in the convection zone of the reactor before being fed into the second vessel.

62. The process as claimed in claim 29, wherein the temperature of the reaction gas entering the heating apparatus located outside the reactor is measured and serves as command variable for regulating the amount of the chemical promoter added and/or for the intensity of the localized energy input.

63. The process as claimed in claim 29, wherein the molar conversion of the dissociation reaction is determined at a location which is either downstream of the heating apparatus for the halogenated aliphatic hydrocarbon or at the top of the quenching column by means of an on-line gas chromatograph.

64. The process as claimed in claim 29, further comprising condensation of the flue gas in at least one heat exchanger and the utilization of the waste heat of the flue gas for preheating the burner air.

65. The process as claimed in claim 64, wherein the flue gas is extracted by means of a flue gas blower after leaving the convection zone and is passed through a heat exchanger where it is condensed, the waste heat is utilized for heating the burner air, the condensate formed is worked up and discharged from the process, the remaining gaseous constituents of the flue gas are purified and are released into the atmosphere.

66. The process as claimed in claim 29, wherein the flue gas to be cooled to below the dew point is introduced in a downward direction into the heat exchanger, after cooling leaves the heat exchanger in an upward direction and wherein the condensate formed can freely run off downward from the heat exchanger and is separated off from the flue gas stream.

67. An apparatus for the thermal dissociation of halogenated aliphatic hydrocarbons to form ethylenically unsaturated halogenated hydrocarbons, which comprises a heated reactor comprising a reaction flow tube having a plurality of tube sections defined therein and adapted for passage of both liquid and gaseous halogenated hydrocarbon therethrough, said heated reactor having a convection zone and a radiation zone, said reaction flow tube passing through said convection zone and said radiation zone, said radiation zone being located downstream from said convection zone when viewed in the flow direction of the reaction gas flow,
said apparatus comprising:
a.) means for introducing an initiator for accelerating the dissociation of said halogenated aliphatic hydrocarbon including:
i.) means of introducing chemical promoters for the thermal dissociation reaction into the reaction tubes;
ii.) means of introducing localized energy to form free radicals in the reaction tubes, or
iii.) a combination of means for introducing chemical promoters and localized energy to form free radicals;
b.) one or more burners located in the radiation zone which heat a section of the reaction tube, and
c.) one or more burners located in the convection zone which heat a section of the reaction flow tube.

68. The apparatus as claimed in claim 67, wherein means for introducing an initiator for accelerating the dissociation of said halogenated aliphatic hydrocarbon include means of introducing chemical promoters for the thermal dissociation into the halogenated aliphatic hydrocarbon into the reaction flow tube located in the radiation zone and comprise feed lines which allow the introduction of predetermined amounts of chemical promoters into the flow of halogenated aliphatic hydrocarbon therethrough.

69. The apparatus as claimed in claim 67, wherein means for introducing an initiator for accelerating the dissociation of said halogenated aliphatic hydrocarbon include means of introducing chemical promoters for the thermal dissociation into the halogenated aliphatic hydrocarbon flow through the reaction flow tube located in the radiation zone opening into the reaction flow tube in the first third of the radiation zone when viewed in the flow direction of the halogenated aliphatic hydrocarbon; and comprise feed lines having nozzles opening into the reaction flow tube.

70. The apparatus as claimed in claim 67, wherein means for introducing an initiator for accelerating the dissociation of said halogenated aliphatic hydrocarbon include means of introducing localized energy to form free radicals in the reaction tubes are located in the radiation zone opening into the reaction flow tube in the first third of the radiation zone when viewed in the flow direction of the halogenated aliphatic hydrocarbon.

71. The apparatus as claimed in claim 70, wherein the apparatus further comprises:
a.) means of determining the dew point of the flue gas, and
b.) means, responsive to changes in the dew point of said flue gas, for regulating the degree of dissociation of halogenated aliphatic hydrocarbon by:
controlling the amount of fuel fed to said burners;
regulating the amount of the chemical promoter added;
regulating the intensity of the localized energy input.

72. The apparatus as claimed in claim 67, further comprising a regulating circuit adapted for selecting the amount of the initiator for accelerating the dissociation of said halogenated aliphatic hydrocarbon introduced into the reaction flow tube, an actuating variable chosen from the group consisting of temperature of the exiting reaction gases, the content of dissociation products in the reaction gases; wall temperature of the reaction flow tube at a point or points and combinations of the foregoing being used in said regulating circuit to regulate the amount of the initiator introduced into the reaction flow stream.

73. The apparatus as claimed in claim 72, wherein an apparatus for vaporizing halogenated aliphatic hydrocarbon is provided which is located outside the reactor and comprises a first vessel and a second vessel, with the liquid halogenated aliphatic hydrocarbon being heated to boiling by the product gas in the first vessel and from there being transferred to the second vessel in which it is partly vaporized without further heating under a pressure which is lower than in the first vessel and the vaporized feed gas is fed into the reactor and unvaporized halogenated aliphatic hydrocarbon is recirculated to the first vessel.

74. The apparatus as claimed in claim 67, wherein an apparatus for vaporizing halogenated aliphatic hydrocarbon is provided which is located outside the reactor and comprises a first vessel and a second vessel, with the liquid halogenated aliphatic hydrocarbon being heated to boiling by the product gas in the first vessel and from there being transferred to the second vessel in which it is partly vaporized without further heating under a pressure which is lower than in the first vessel and the vaporized feed gas is fed into the reactor and unvaporized halogenated aliphatic hydrocarbon is recirculated to the first vessel.

75. The apparatus as claimed in claim 74, wherein before being fed into the second vessel, halogenated aliphatic hydrocarbon is conveyed through the convection zone of the reactor where it is heated by means of the flue gas produced by the burners which heat the reactor.

76. The apparatus as claimed in claim 67, wherein one portion of the reaction flow tube located in the radiation zone of said furnace shields another portion of said reaction flow tube located thereabove in the convection zone from direct radiation from the radiation zone and wherein said burners which heat the reaction tubes in the convection zone are arranged above the shock tubes.

77. The apparatus as claimed in claim 67, wherein the apparatus further comprises:
a.) means of determining the dew point of the flue gas, and
b.) means, responsive to changes in the dew point of said flue gas, for regulating the degree of dissociation of halogenated aliphatic hydrocarbon by:
controlling the amount of fuel fed to said burners;
regulating the amount of the chemical promoter added;
regulating the intensity of the localized energy input.
